(19) United States
(12) Patent Application Publication
(10) Pub. No.: US 2014/0112983 A1
(43) Pub. Date: Apr. 24, 2014

(54) NITRITE COMPOSITIONS AND USES THEREOF

(71) Applicants: Anthony Giordano, Chesterland, OH (US); Christopher Kevil, Shreveport, LA (US); Paul Sonz, Belle Air Bluffs, FL (US); James Douglas Boudreaux, Shreveport, LA (US)

(72) Inventors: Anthony Giordano, Chesterland, OH (US); Christopher Kevil, Shreveport, LA (US); Paul Sonz, Belle Air Bluffs, FL (US); James Douglas Boudreaux, Shreveport, LA (US)

(73) Assignee: TheraVasc Inc., Cleveland, OH (US)

(21) Appl. No.: 14/009,927

(22) PCT Filed: Apr. 13, 2012
(86) PCT No.: PCT/US12/33520

§ 371 Date: Jan. 9, 2014

Related U.S. Application Data
(60) Provisional application No. 61/475,582, filed on Apr. 14, 2011.

(51) Int. Cl. A61K 33/00; A61K 36/16; A61K 36/88; A61K 36/9068; A61K 36/67; A61K 31/145; A61K 36/752; A61K 36/74; A61K 36/481; A61K 36/61; A61K 36/79; A61K 45/06; A61K 36/23

(52) U.S. Cl. CPC A61K 33/00; A61K 45/06; A61K 36/16; A61K 36/88; A61K 36/9068; A61K 36/67; A61K 36/23; A61K 36/752; A61K 36/74; A61K 36/481; A61K 36/61; A61K 36/79; A61K 31/145
USPC 424/457; 424/718

(57) ABSTRACT

The present invention relates to compositions of nitrite and methods for prophylactic nutritional supplementation and therapeutic nutritional supplementation. Specifically, the method involves administering to an individual a composition of inorganic nitrite, or a pharmaceutically acceptable salt thereof, for supplementation in subjects with diabetes, peripheral artery diseases or in patients with risk factors associated with cardiovascular diseases.

### CROSS-REFERENCE TO RELATED APPLICATIONS

This application claims benefit of U.S. Provisional Application No. 61/475,582, filed Apr. 14, 2011, which is hereby incorporated by reference in its entirety.

### FIELD OF THE INVENTION

The present invention relates to dietary supplements containing nitrite salts and nutritional uses thereof.

The present invention also relates to compositions of inorganic nitrite, and pharmaceutically acceptable salts of said inorganic nitrite, and methods for using these compositions for prophylactic nutritional supplementation and therapeutic nutritional supplementation in, for example, cardiovascular and vascular disease conditions.

### BACKGROUND OF THE INVENTION

Nutrition plays a critical role in maintaining good health, preventing dietary deficiencies, and protecting against physiological stress and the development of disease. For example, as the body ages it suffers significant physiological stresses. Specifically, as the body metabolism shifts to accumulating larger fat stores and decreasing lean body mass, this increase in body weight may lead to obesity and associated conditions such as diabetes, cardiovascular disease, peripheral artery disease, chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, or peripheral neuropathy and stem cell diseases. In particular, these disease states may result in decreased circulating nitrite levels or decreased nitric oxide (NO) levels that further compromise health.

Nitric oxide has been shown to positively regulate endothelial cell responses, can stimulate tissue regeneration, enhance stem cell growth and proliferation and alter inflammatory responses. NO increases the expression of various angiogenic factors which, together with other mediators, increases NO levels via a positive feedback mechanism. In addition to stimulating the growth of endothelial cells, NO can also protect tissues against ischemic damage by slowing cellular respiration. NO has been shown to modulate several endothelial cell signaling pathways for example, Erk 112 and PKC.

The primary enzyme responsible for NO production in the cardiovascular system is endothelial nitric oxide synthase (eNOS) which is regulated by numerous molecules and signaling pathways. Importantly, eNOS activity is also largely responsible for systemic NO production as the amount of enzyme expression is often directly proportional to NO metabolite levels. NO readily diffuses across lipid bilayers and its biological fate is dictated predominately by reactions with metalloproteins and other free radical species; the classic example being activation of the heme enzyme soluble guanylate cyclase (sGC) which initiates a signal cascade leading to vessel dilation and platelet inhibition. In addition, NO may also be oxidized through various mechanisms resulting in the formation of nitrite which can be further oxidized to nitrate.

Both nitrite and nitrate are involved in regulating production of NO from NOS independent pathways. Inorganic nitrite can undergo a one electron reduction back to NO through various mechanisms with oxygen-binding heme proteins (hemoglobin and myoglobin), deoxyhemoglobin, deoxymyoglobin, xanthine oxidoreductase, endothelial nitric oxide synthase, acidic disproportionation, and members of the mitochondrial electron transport chain, e.g., mitochondrial heme proteins all being potential electron donors. The ability of nitrite to be reduced back to NO classifies it as a unique NO donor under biological conditions, e.g., tissue ischemia, in which many of these potential reducing agents are active.

Plasma nitrite levels have been shown to be inversely correlated to cardiovascular risk factors, with subjects having the greatest number of risk factors, having the lowest level of plasma nitrites (Kleinbongard et al., 2006, Free Radical Biology & Medicine 40: 295-302). In addition, in normal subjects, exercise results in a release of stored nitrite to the plasma, increasing plasma nitrite levels.

However, in diabetic and PAD patients, exercise does not increase the level of plasma nitrite and in fact, leads to a further decrease in circulating nitrite levels (Allen et al., 2009, Nitric Oxide 20:231-237).

Thus, nutritional supplementation serves a vital role in protecting against poor nutrition and disease. More specifically, nutritional supplementation may provide the nutrients that might otherwise be lacking in the diet, and provide the nutritional defense against disease development. Similarly, nutritional supplementation may provide the nutrients that are deficient in many disease states.

### BRIEF SUMMARY OF THE INVENTION

In a first aspect, the invention features a dietary supplement including one or more fruit or herbal extracts and at least 5% by weight of a nitrite salt

In some embodiments, the extract is an herbal extract selected form the group consisting of gingko biloba, garlic, ginger, pepper, parsley, orange, yohimbine, astragalus, catuaba, schisandra, and sulforaphane.

In certain embodiments, the herbal extract is a mixture of gingko biloba and garlic.

In other embodiments, the herbal extract is gingko biloba.

In some embodiments, the dietary supplement is formulated for steady-state release over a period of about 2 hours to about 24 hours.

In some embodiments, the dietary supplement is formulated for daily administration

In some embodiments, the dietary supplement is formulated for oral administration.

In certain embodiments, the nitrite salt is sodium nitrate, potassium nitrite, or calcium nitrite.

In other embodiments, the dietary supplement is in the form of a tablet or capsule.

In another aspect, the invention features a method for increasing nitrite levels in a person having anitrite nutritional deficiency where the method includes administering to the person any of the dietary supplements described herein in a dosing regimen sufficient to alleviate the nutritional deficit.

In some embodiments, the dosing regimen administered to a subject results in a plasma level of nitrite of about 0.05 to 0.6 μM.

In other embodiments, a unit dose from 0.5 μg/kb to about 1000 μg/kg is administered.

In other embodiments, the nitrite nutritional deficiency is a result of dietary restrictions.

In some embodiments, the nitrite nutritional deficiency is a result of a disease state.

In certain embodiments, the disease state is selected from the group consisting of diabetes, peripheral artery disease, chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, peripheral neuropathy, and stem cell diseases, or a combination of one or more of the disease states.

In still other embodiments, the disease state leads to decreased circulating levels of nitrite.

In some embodiments, the disease state leads to decreased stored levels of nitrite.

In further embodiments, the disease state leads to decreased circulating levels of nitric oxide.

In another aspect, the invention features an oral pharmaceutical composition for treating nitrite deficiency in a subject, which composition includes a sufficient amount of a pharmaceutically acceptable nitrite salt so that upon administration an increased serum nitrite concentration of from about 200 nM to about 1000 nM of nitrite is achieved.

In some embodiments, the oral pharmaceutical composition further includes an enteric coating.

In other embodiments, the enteric coating serves to release the nitrite salt to achieve the increased serum nitrite concentration over a period of at least 8 hours.

In still other embodiments, the enteric coating serves to release the nitrite salt to achieve the increased serum nitrite concentration over a period of at least 6 hours.

In further embodiments, the enteric coating serves to release the nitrite salt to achieve the increased serum nitrite concentration over a period of at least 4 hours.

In certain embodiments, the enteric coating serves to release the nitrite salt to achieve the increased serum nitrite concentration over a period of at least 2 hours.

In other embodiments, the oral pharmaceutical composition increases the serum nitrite concentrations to least 300 nM or at least 400 nM.

In some embodiments, the pharmaceutically acceptable nitrite salt is selected from the group consisting of sodium nitrite, potassium nitrite, and calcium nitrite, or mixtures thereof.

In certain embodiments, the oral pharmaceutical composition further includes a compound selected from a sulfide, clopidogrel, or dipyridamole.

In still another aspect, the invention features a method for treating nitrite deficiency in a subject, which method includes administering to the subject any of the oral pharmaceutical compositions described herein.

In some embodiments, the oral pharmaceutical composition is administered to the subject in a pharmaceutically acceptable nitrite salt to subject weight/weight ratio of from about 0.1 to about 2 mg/kg.

In other embodiments, treatment is continued until the tissue concentration of nitrite in the patient reaches between 300 nM and 1000 nM.

In still other embodiments, the composition is administered in a form to provide a steady-state release over a period of about 2 hours to about 24 hours.

In another aspect, the invention features a method for supplementing nutritional deficiencies in a patient or person in need thereof, including administering to a patient or a person a composition of inorganic nitrite or a pharmaceutically acceptable salt thereof, where the nitrite is administered for a time and in an amount sufficient to correct the nutritional deficits found in subjects.

In some embodiments, the dose administered to a patient results in a plasma level of nitrite of about 0.1 to 1.0 μM, preferably, 0.3 μM, and which does not lead to an increase in the ration of methemoglobin:globin of more than 1:10 in the patient.

In other embodiments, the dose is about 100 μg/kg to about 2000 μg/kg.

In still other embodiments, the composition is administered to the patient daily.

In certain embodiments, the composition is administered to the patient orally.

In other embodiments, the composition is administered to the patient, sublingually, topically, intramuscularly, intraperitoneally, intravenously, or subcutaneously.

In still other embodiments, the composition is administered in a form to provide a steady-state release over a period of about 2 hours to about 24 hours.

In certain embodiments, the composition further includes a pharmaceutically acceptable carrier.

In some embodiments, the pharmaceutically acceptable salt of inorganic nitrite is sodium nitrite, potassium nitrite, or calcium nitrite.

In other embodiments, nutritional deficiencies are a result of dietary restrictions.

In still other embodiments, the nutritional deficiencies are a result of a disease state.

In some embodiments, the disease state is selected from one or more of the group consisting of diabetes, peripheral artery disease, chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, or peripheral neuropathy and stem cell diseases.

In other embodiments, the disease state leads to decreased circulating levels of nitrite in the patient.

In certain embodiments, the disease state leads to decreased stored levels of nitrite in the patient.

In still other embodiments, the disease state leads to decreased stored levels of nitrite in the patient.

In some embodiments, the disease states leads to decreased circulating levels of nitric oxide in the subject.

In any of the methods described herein, the method can also include the administration of a compound selected from a sulfide, clopidogrel, or dipyridamole

As used in the specification and claims, the singular form “a”, “an” and “the” include plural references unless the context clearly dictates otherwise.

All numerical designations, e.g., pH, temperature, time, and concentration, and including ranges, are approximations which are varied (+) or (−) by increments of 5%. It is to be understood, although not always explicitly stated that all numerical designations are preceded by the term “about”.

The term “subject” refers to an animal, preferably a mammal and includes nonprimates and primates (e.g. monkeys, chimpanzees, and humans). Animals also include dogs, livestock (e.g. horses, cattle, sheep, pigs) and laboratory animals such as rodents (mice, rats, etc.), rabbits, and guinea pigs.

The term “nitrite” refers to NO₂⁻, the conjugate base of nitrous acid HNO₂. Nitrite salts refers to salts formed from the nitrite ion and a cationic metal such as alkali and alkaline earth metals. Suitable salts include potassium, sodium, and calcium (KNO₂, NaNO₂, and Ca(NO₂)₂) or mixtures thereof. Nitrite salts are readily available commercially and methods for preparing nitrite salts are well known in the art. Nitrite salts can be synthesized by reacting a mixture of nitrogen monoxide (NO) and nitrogen dioxide (NO₂) with a corresponding metal hydroxide solution, as well as through the thermal decomposition of the corresponding nitrate.

The term “fruit extract(s)” refers to natural extract(s) isolated from fruits.

The term “herbal extract(s)” refers to natural extract(s) isolated from plants and plant parts such as roots and seeds. The herbal extracts may be isolated from plants such as seaweed and kelp.

Preferred fruit and herbal extracts include those that promote tissue perfusion, blood flow, and/or vasodilation.

As used herein, the term “delayed release” refers to a pharmaceutical preparation, e.g., an orally administered formulation, which passes through the stomach substantially intact and dissolves in the small and/or large intestine (e.g., the colon). In some embodiments, delayed release of the active agent (e.g., nitrite as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably refer to a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” refer to pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “pharmaceutical composition,” as used herein, represents a composition containing a compound described herein (e.g., inorganic nitrite, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, represents those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, means a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prodrug,” as used herein, represents compounds which are rapidly transformed in vivo to the parent compound of the above formula. Prodrugs also encompass bioequivalent compounds that, when administered to a human, lead to the in vivo formation of nitrite ion (NO₂⁻) or nitrous oxide (NO). A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable such as those described in EP 1336602A1, which is herein incorporated by reference.

As used herein, and as well understood in the art, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also refer to delaying the onset of, retarding or reversing the progress of, or alleviating either the disease or condition to which the term applies, or one or more symptoms of such disease or condition.

The term “unit dosage forms” refers to physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” refers to the amount of nitrite ion present in the plasma of a treated subject (e.g., as measured in a rabbit using an assay described below or in a human).

Other features and advantages of the invention will be apparent from the following Detailed Description, the drawings, and the claims.

### DETAILED DESCRIPTION OF THE INVENTION

### Nitrite

Inorganic Nitrite

The pharmaceutically acceptable compositions of the invention include inorganic nitrite, e.g., a salt or ester of nitrous acid (HNO₂), or a pharmaceutically acceptable salt thereof. Nitrite salts can include, without limitation, salts of alkali metals, e.g., sodium, potassium; salts of alkaline earth metals, e.g., calcium, magnesium, and barium; and salts of organic bases, e.g., amine bases and inorganic bases.

Compounds of the invention also include all isotopes of atoms occurring in the intermediate or final compounds. Isotopes include those atoms having the same atomic number but different mass numbers. For example, isotopes of hydrogen include tritium and deuterium.

The term “compound,” as used herein with respect to any inorganic nitrite or pharmaceutically acceptable salt, solvate, or prodrug thereof. All compounds, and pharmaceutical acceptable salts thereof, are also meant to include solvated (e.g., hydrated) forms. Nitrite has the chemical formula NO₂⁻ and may exist as an ion in water. Sodium nitrite has the chemical formula NaNO₂ and typically dissolves in water to form the sodium ion Na⁺ and the nitrite ion NO₂⁻. It will further be understood that the present invention encompasses all such solvated forms (e.g., hydrates) of the nitrite compounds. Exemplary nitrite compounds are described in WO 2008/105730, which is hereby incorporated by reference.

In addition to sodium nitrite, representative inorganic nitrite compounds include: ammonium nitrite (NH₄NO₂), barium nitrite (Ba(NO₂)₂; e.g., anhydrous barium nitrite or barium nitrite monohydrate), calcium nitrite (Ca(NO₂)₂; e.g., anhydrous calcium nitrite or calcium nitrite monohydrate), cesium nitrite (CsNO₂), cobalt(II)nitrite (Co(NO₂)₂), cobalt(III)potassium nitrite (CoK₃(NO₂)₆; e.g., cobalt(III)potassium nitrite sesquihydrate), lithium nitrite (LiNO₂; e.g., anhydrous lithium nitrite or lithium nitrite monohydrate), magnesium nitrite (MgNO₂; e.g., magnesium nitrite trihydrate), potassium nitrite (KNO₂), rubidium nitrite (RbNO₂), silver(I)nitrite (AgNO₂), strontium nitrite (Sr(NO₂)₂), and zinc nitrite (Zn(NO₂)₂).

The compounds of the present invention can be prepared in a variety of ways known to one of ordinary skill in the art of chemical synthesis. Methods for preparing nitrite salts are well known in the art and a wide range of precursors and nitrite salts are readily available commercially. Nitrites of the alkali and alkaline earth metals can be synthesized by reacting a mixture of nitrogen monoxide (NO) and nitrogen dioxide (NO₂) with a corresponding metal hydroxide solution, as well as through the thermal decomposition of the corresponding nitrate. Other nitrites are available through the reduction of the corresponding nitrates.

The present compounds can be prepared from readily available starting materials using the methods and procedures known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one of ordinary skill in the art by routine optimization procedures.

Suitable pharmaceutically acceptable salts include, for example, sodium nitrite, potassium nitrite, or calcium nitrite. Still other exemplary salts are found in Remington's Pharmaceutical Sciences, 17th ed., Mack Publishing Company, Easton, Pa., 1985, p. 1418, Berge et al., J. Pharmaceutical Sciences 66:1-19, 1977 and Pharmaceutical Salts: Properties, Selection, and Use, (Eds. P. H. Stahl and C. G. Wermuth), Wiley-VCH, 2008, each of which is incorporated herein by reference in its entirety.

### Pharmaceutical Compositions

The methods described herein can also include the administrations of pharmaceutically acceptable compositions that include inorganic nitrite, e.g., a salt of nitrous acid (HNO₂) such as NaNO₂, or a pharmaceutically acceptable salt, solvate, or prodrug thereof (e.g., nitrates). When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intraarterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary). In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 6^{th} Edition, Rowe et al., Eds., Pharmaceutical Press (2009).

The methods described herein can include the admnitration of nitrate salts, or prodrugs or pharmaceutical compositions thereof, or other therapeutic agents. Exemplary nitrate salts are described in WO 2008/105730. Exemplary therapeutic agents that may be included in the compositions described herein are cardiovascular therapeutics (e.g., anti-thrombotics (e.g. dipyridamole, clopidogrel, and the like), anti-hypertensives (e.g., Ca⁺⁺ channel blockers, AT-2 blockers, ACE inhibitors, and the like), anti-cholesterols (e.g., statins, fibrates, and the like), and thiazolidinedione therapeutics.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992)

Oral transmucosal absorption is generally rapid because of the rich vascular supply to the mucosa and allows for a rapid rise in blood concentrations of the therapeutic (“American Academy of Pediatrics: Alternative Routes of Drug Administration—Advantages and Disadvantages (Subject Review),” Pediatrics, 100(1):143-152, 1997).

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

### Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce chronic tissue ischemia.

### Dietary Supplements

The present invention provides dietary supplements for nutritional supplementation in subjects having a nitrite nutritional deficiency resulting from dietary restrictions or a disease state. In one embodiment, provided is a dietary supplement comprising one or more fruit or herbal extracts and at least 5% by weight of a nitrite salt.

A dietary supplement provided herein can contain any number of different extracts. For example, the dietary supplement may contain one, two, three, four, five, six, seven, eight, nine, ten, or more different extracts.

The extracts can independently be in any amounts, such as for example up to 0.0001%, 0.001%, 0.01%, 0.1%, 0.5%, 1%, 3%, 5%, 10%, 15%, 20%, 25%, 30%, 35%, 40%, 45%, 50%, 55%, 60%, 65%, 70%, 75%, 80%, 85%, 90%, or 95% by weight of the supplement. For example, the dietary supplement may contain extracts in the amounts of 1 mg to 5 gm.

The extracts can be in any form such as, for example, a solution, powder, or soluble powder.

Extracts may be obtained from commercial sources or isolated from natural sources using known methods. Such methods include drying of the fruit, plant, or plant part followed by extraction of the desired constituent by using a variety of techniques such as organic solvent extraction, distillation, filtration, and chromatography.

In one embodiment, provided is a dietary supplement containing at least one herbal extract selected from the group consisting of gingko biloba, garlic, ginger, pepper, parsley, orange, yohimbine, astragalus, catuaba, schisandra, and sulforaphane. In other embodiments, the herbal extract is gingko biloba or garlic.

The dietary supplements are formulated in solution, suspension, powder, or solid dosage form. Solid dosage forms include pills such as tablets and capsules.

In some embodiments, the solid dosage forms are formulated so as to maximize absorption of the nitrite salts in the small intestine (duodenum, jejunum, ileum) and minimize contact of the salts with the gastric acids. The solid dosage forms may contain pH-sensitive or enzyme sensitive coatings that disintegrate maximally when in contact with intestinal pH and/or enzymes. The coatings release the nitrite salts by disintegration or by becoming porous to allow for dispersion of the salts.

Pellets and granules containing the nitrite salt and extract can have sustained or controlled release properties and can be prepared with enteric coatings using conventional methods (e.g. spinning disc, spray drying, etc.) or by using melt granulation techniques with waxes or silicone elastomers. The pellets and granules can then be filled into capsules or compressed into tablets. When so formulated, the enterically coated pellets or granules may be released into the stomach, with the salts themselves released in the small intestine when the pellets or granules are exposed to intestinal fluids. Different coatings or coatings with varying thicknesses may also be used for the pellets or granules within the same dosage form, so that dissolution varies and occurs with increasing intestinal pH as the dosage form travels along the intestinal tract. The tablets or capsules themselves containing nitrite salts or enterically coated nitrite pellets/granules may also be enterically coated (such as by dip coating) so as to release their contents in the intestines.

The polymers used to enteric coat a tablet, capsule, or pellet may be selected from the group of anionic carboxylic polymers suitable for pharmaceutical purposes and being soluble only with difficulty at a low pH but being soluble at a higher pH, the pH limit for solubility being in the interval of pH 4 to pH 7.5. Exemplary, non-limiting polymers include shellac, cellulose acetate phthalate (for example, Aquateric; FMC Corporation, Pharmaceutical Division, 1735 Market Street, Philadelphia, Pa. 19103), cellulose acetate trimellitate, hydroxypropylmethycellulose phthalate, polyvinyl acetate phthalate, carboxymethyl ethylcellulose, and acrylic acid polymers (e.g., Acryl-EZE (Colorcon) and partly esterified methacrylic acid polymers such as Eudragit L, Eudragit L 100-55, and Eudragit S. These polymers may be used alone or in combination with each other.

The enteric coatings may optionally comprise other pharmaceutically acceptable materials which improve the properties of the film-forming polymers such as plasticizers, anti-adhesives, surfactants, and diffusion-accelerating or diffusion-retarding substances, and may also comprise fillers, pigments, and buffers. Suitable plasticizers comprise phthalic acid esters, triacetin, dibutylsebacate, monoglycerides, citric acid esters and polyethyleneglycols. Preferred plasticizers are acetyltributyl citrate and triethyl citrate. Suitable anti-adhesives comprise talc and metal stearates.

The capsule may be a gelatin capsule (for example, a capsule which consists essentially of gelatin) which may then be enteric coated. Suitable capsules are well known to those skilled in the art. It is intended that the capsules are such that they release their liquid or solid contents at least in the stomach or in the intestines and are not meant to pass through the intestinal tract intact.

The solid dosage forms may contain disintegrants such as starches, clays, celluloses, algins, gums, and cross-linked polymers that aid in dissolution. Disintegrants also include corn and potato starch, Veegum HV, methylcellulose, agar, bentonite, cellulose and wood products, natural sponge, cation-exchange resins, alginic acid, guar gum, citrus pulp and carboxymethylcellulose, Croscarmelose (a cross-linked cellulose), crospovidone (a cross-linked polymer), sodium starch glycolate (a cross-linked starch), cross-linked PVP, and acdisol. A preferred disintegrant is microcrystalline cellulose such as Avicel (e.g., Avicel PH101, PH102, PH301, and PH302; FMC Corporation, Pharmaceutical Division, 1735 Market Street, Philadelphia, Pa. 19103).

In one embodiment, the nitrite salts comprise at least 5% by weight of the dietary supplement. In other embodiments, the nitrite salts comprise at least 10%, 15%, 20%, 25%, 30%, 35%, 40%, 45%, 50%, 55%, 60%, 65%, 70%, 75%, 80%, 85%, 90%, or 95% by weight of the supplement. Such supplement can be formulated in a unit dosage form, each dosage containing, for example, from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about I mg, from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg, from about 1 mg to about 50 mg, from about 1 mg to about 30 mg, from about I mg to about 20 mg, from about 1 mg to about 10 mg, from about 1 mg to about 5 mg; from about 5 mg to about 50 mg, from about 5 mg to about 20 mg, from about 5 mg to about 10 mg, from about 10 mg to about 100 mg, from about 20 mg to about 200 mg, from about 30 mg to about 150 mg, from about 40 mg to about 100 mg, and from about 50 mg to about 100 mg of the nitrite salt.

The amount of inorganic nitrite per dose can vary. For example, a subject can receive from about 0.05 μg/kg to about 10000 μg/kg., e.g., about 0.05, 1, 5, 10, 25, 50, 100, 200, 250, 300, 400, 450, 500, 550, 600, 650, 700, 750, 800, 850, 900, 1000, or 2000 μg/kg. Exemplary dosages include 0.1-5000 μg/kg, 0.5-1000 μg/kg, 100-1500 μg/kg, 100-350 μg/kg, 340-750 μg/kg, 350-750 μg/kg, or 750-1000 μg/kg.

The frequency of supplementation may also vary. The subject can be supplemented one or more times per day (e.g., once, twice, three, four or more times) or every so-many hours (e.g., about every 2, 4, 6, 8, 12, or 24 hours). The time course of supplementation may be of varying duration, e.g., for two, three, four, five, six, seven, eight, nine, ten or more days. For example, the supplementation can be twice a day for three days, twice a day for seven days, twice a day for ten days. Supplementation cycles can be repeated at intervals, for example weekly, bimonthly or monthly, which are separated by periods in which no supplement is given. The supplement can be a single supplement or can last as long as the life span of the subject (e.g., many years). In one embodiment, the compositions provided herein are taken by the subject just prior to exercising to supplement the nutritional deficiencies observed during exercise.

In one aspect, the solid dosage forms provide herein release the nitrite salt to achieve the increased serum nitrite concentration over a period of at least 4, 3, 2, or 1 hour(s). In some such aspects, the solid dosage forms release the nitrite salt within 10 minutes upon contact with gastric fluids. In other aspects, the serum nitrite concentration is maintained for at least 24, 18, 16, 14, or 12 hours when the does is administered once, twice, or three times within a 24 hour time period.

The oral solid dosage forms provided herein increase serum nitrite concentration of from about 200 nM to about 500 nM upon administration. In some aspects, the compositions increase serum nitrite concentrations to least 300 nM. In other aspects, the serum nitrite concentrations are increased to at least 400 nM.

In one embodiment, the present methods for supplementing nutritional deficits are carried out by administering an inorganic nitrite for a time and in an amount sufficient to result in an increase in plasma nitrite or plasma nitric oxide levels, without increasing methemoglobin levels to more than about 10% of the total globin content. The amount and frequency of administration of the compositions can vary depending on, for example, what is being administered and the state of the patient's nutritional deficit. The dosage is likely to depend on such variables as the type and extent of nutritional deficit, the age, weight and general condition of the particular patient, and the judgment of the attending clinician. Effective doses can be extrapolated from dose response curves derived from in vitro or animal model test system. An effective dose is a dose that produces a desirable increase in plasma levels of nitrite or nitric oxide without increasing the ratio of methemoglobin:globin above 1:10.

In one embodiment, the dietary supplement provides a sustained release of nitrite such that circulating plasma levels of nitrite in a range of about 0.2 μM (200 nM) to about 0.5 μM (500 nM) is achieved, and such that methemoglobin:globin ratios of less than 1:10 are produced by the supplements. In some aspects, the dosing regimen administered to a subject results in a plasma level of nitrite of about 0.05 to 0.6 μM, preferably, 0.3 μM, and which does not lead to a methemoglobin:globin ratio of more than 1:10.

The dietary compositions may be used to supplement the nutritional deficiencies of subjects suffering from a disease state that results in decreased plasma nitrite or nitric oxide levels. Nutritional deficiencies have been observed in subjects with risk factors associated with cardiovascular diseases and in peripheral artery disease and diabetic subjects. These deficiencies are further pronounced in the latter subject groups following exercise.

Other subjects which may also benefit from nutritional supplementation with nitrite salts include those with chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, peripheral neuropathy, or stem cell diseases, and subjects with dietary restrictions.

### Nutritional Compositions

The present invention provides nutritional compositions of inorganic nitrite for both prophylactic and therapeutic nutritional supplementation, specifically in cardiovascular, metabolic, inflammatory or vascular diseases. Specifically, the present invention relates to novel compositions of inorganic nitrite, or an acceptable pharmaceutical salt thereof, that can be used to supplement the nutritional deficiencies observed in patients with diabetes, peripheral artery disease, chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, or peripheral neuropathy, stem cell diseases, and/or dietary restrictions. In addition, the compositions may be used to treat the nutritional deficiencies of patients suffering from a disease state that results in decreased plasma nitrite or nitric oxide levels.

Plasma nitrite levels have been shown to be inversely correlated to cardiovascular risk factors, with subjects having the greatest number of risk factors, having the lowest level of plasma nitrites (Kleinbongard et al., 2006, Free Radical Biology & Medicine 40: 295-302). In addition, in normal subjects, exercise results in a release of stored nitrite to the plasma, increasing plasma nitrite levels, however, in diabetic and PAD patients, exercise docs not increase the level of plasma nitrite and in fact, leads to a further decrease in circulating nitrite levels (Allen et al., 2009, Nitric Oxide 20:231-237). Thus, a nutritional supplementation of nitrite might be effective in overcoming these deficits in plasma nitrite levels in cardiovascular and vascular disorders and given the relationship of nitrite to nitric oxide, the deficits in nitric oxide found in these diseases or due to dietary deficiencies in nitrite.

Nitrites are found in many foods (White, 1975, J. Agri. Food Chem. 23:886-891) and dietary supplementation can restore plasma nitrite and nitric oxide levels (Kanematsu, 2008, Am. J. Physiol. Renal. Physiol. 295:FI457-F1462). While nitrite has been used therapeutically for centuries, it has generally been administered acutely to treat such conditions as hypotension and more recently cyanide poisoning. However, concerns about high levels of nitrites leading to nitrosamine production, which in turn promotes cancer formation, and to production of methemeglobinemia, which if severe enough can result in a coma and death, have limited the use of nitrites. As a nutritional supplement, a level of nitrite must be administered to that results in normal plasma levels while not reaching levels that might lead to dangerous levels of methemeglobinemia. The present invention describes such nutritional supplements.

The compositions of the present invention include various formulations of inorganic nitrite, or a pharmaceutically acceptable salt thereof, that improve the nutritional state of a patient; these compositions preferably may be used therapeutically or prophylactically. The pharmaceutically acceptable compositions of the invention include inorganic nitrite, e.g., a salt or ester of nitrous acid (HNO₂) or a pharmaceutically acceptable salt thereof.

In a preferred embodiment, the compositions of the present invention are administered in a manner such that said compositions are formulated to provide sustained released over the course of 2-24 hours, providing consistent supplementation of the nutritional deficiency. Various methods of sustained release are known to those skilled in the art, and can be used for oral formulations of the compositions. Further, nanoparticles and other matrices can be used and implanted to provide sustained release of the composition for much longer periods of time, from about 24 hours to about five years.

In addition, the compositions of the present invention may be administered to an individual on a daily basis for supplementing nutritional deficiencies and the composition may be administered orally. Other means of administering the composition include sublingually, topically, intramuscularly, intraperitoneally, intravenously or subcutaneously. Furthermore, the compositions of the present invention may include a pharmaceutically acceptable carrier.

In a further preferred embodiment, compositions of the present invention are delivered such that the circulating concentration of nitrite does not exceed 0.6 μM (i.e., the nitrite is administered in a dose sufficient to produce a circulating concentration of nitrite in the subject that does not exceed 0.6 μM). For example, the nitrite can be administered in an amount such that the circulating concentration does not exceed 0.05 μM, 0.1 μM, 0.15 μM, 0.2 μM, 0.25 μM, 0.3 μM, 0.35 μM, 0.4 μM, 0.45 μM, 0.5 μM 0.55 μM, or 0.5 μM. Thus, exemplary dosages can produce a circulating concentration of nitrite in the subject of up to or up to about 0.3 μM, 0.1 μM, or 0.05 μM. Dosages should be administered such that methemoglobin:globin ratios of less than 1:10 are produced by the compositions.

In another embodiment of the present invention, the composition is provided in a dose from about 0.05 μg/kg up to about 1000 μg/kg., e.g., about 0.05, 1, 5, 10, 25, 50, 100, 200, 250, 300, 400, 450, 500, 550, 600, 650, 700, 750, 800, 850, 900, or 1000 μg/kg. For example, a subject can receive up to or up to about 165 μg/kg, 16.5 μg/kg, or 8.25 μg/kg. Dosages should be administered such that methemoglobin:globin ratios of less than 1:10 are produced by the compositions.

In yet another preferred embodiment, compositions are taken by the subject just prior to exercising to supplement the nutritional deficiencies observed during exercise. The dose should be administered to provide rapid uptake into the plasma.

The methods of the present invention may be used to treat the nutritional deficits found in patients suffering from diabetes, peripheral artery disease, chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, or peripheral neuropathy, stem cell diseases and dietary restrictions. In addition, these methods may be used to treat the nutritional deficiencies in patients suffering from a disease state that results in decreased levels of circulating nitrite or nitric oxide.

In a preferred embodiment, the methods of the present invention utilize compositions comprising inorganic nitrite, or a pharmaceutically acceptable salt thereof, providing a sustained release of the composition to supplement nutritional deficiencies, such that circulating plasma levels of nitrite in a range of about 0.05 μM to about 0.6 μM is achieved, and such that methemoglobin:globin ratios of less than 1:10 are produced by the compositions.

In a further preferred embodiment, the methods of the present invention utilize a composition comprising inorganic nitrite, or a pharmaceutically acceptable salt thereof, providing rapid release of the composition to supplement nutritional deficiencies occurring daily or during exercise, at dosages of about 0.5 μg/kg to about 1000 μg/kg, such that the nutritional deficit is relieved, and such that methemoglobin:globin ratios of less than 1:10 are produced by the compositions. The composition can be administered one or more times each day, preferably orally, although other routes of administration are also acceptable.

Proper nutrition is essential for maintaining health and preventing diseases. The compositions and methods of the present invention provide the means to optimize good health by utilizing inorganic nitrites, or pharmaceutically acceptable salts thereof. More specifically, the compositions of the present invention can be used to correct the nutritional deficits found in patients suffering from diabetes, peripheral artery disease, chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, or peripheral neuropathy, stem cell diseases and dietary restrictions. In addition, these compositions and methods may be used to treat the nutritional deficiencies in patients suffering from a disease state that results in decreased levels of circulating nitrite or nitric oxide.

The compositions and methods of the present invention may preferably by formulated in a manner to provide sustained release of inorganic nitrite, or a pharmaceutically acceptable salt thereof, such that circulating levels of nitrite are achieved to supplement the nutritional deficiency but are not high enough to increase the levels of methemoglobin to more that 10% of the total globin levels. The supplementation of plasma nitrite levels in subjects may also lead to supplementation of plasma nitric oxide levels in these subjects.

The compositions of the present invention may be in the form of a solid powder, caplets, tablets, lozenges, pills, capsules, or a liquid, and which may be administered alone or in suitable combination with other nutritional components. For example, the composition of the present invention may be administered in one or more caplets or lozenges as practical for ease of administration. Other means of delivery, including sublingually, topically, intramuscularly, intraperitoneally, intravenously or subcutaneously are also acceptable.

To prepare the compositions of the present invention, active ingredient, inorganic nitrite, or a pharmaceutically acceptable salt thereof, may be combined in intimate admixture with a suitable carrier according to conventional compounding techniques. The carrier may take a wide variety of forms depending upon the form of the preparation desired for administration, e.g., oral, sublingual, nasal, topical patch, or parenteral. Preferably, the composition may consists of one sustained released capsule or one or more rapid release capsules. The capsule can be provided with or without coating to allow for absorption of the composition in the stomach or intestines. For example, an enteric coating can be applied to the capsule, tablet, caplet, or pill to allow for absorption in the intestines.

In preparing the composition in oral dosage form, any of the usual media may be utilized. For liquid preparations (e.g., suspensions, elixirs, and solutions), media containing, for example water, oils, alcohols, flavoring agents, preservatives, coloring agents and the like may be used. Carriers such as starches, sugars, diluents, granulating agents, lubricants, binders, disintegrating agents and the like may be used to prepare oral solids (e.g., powders, caplets, pills, tablets, capsules, and lozenges). Controlled release forms may also be used. Because of their ease in administration, caplets, tablets, pills, and capsules represent the most advantageous oral dosage unit form, in which case solid carriers are employed. If desired, tablets may be sugar coated or enteric coated by standard techniques. All of these pharmaceutical carriers and formulations are well known to those of ordinary skill in the art. See, e.g., Wade & Waller, Handbook Of Pharmaceutical Excipients (2nd ed. 1994).

### Pharmaceutical Compositions

The pharmaceutically acceptable compositions of the invention include inorganic nitrite, e.g., a salt or ester of nitrous acid (HNO₂) or a pharmaceutically acceptable salt thereof. The nitrite ion is NO₂⁻. More generally, a nitrite compound is either a salt or an ester of nitrous acid. Nitrite salts can include, without limitation, salts of alkali metals, e.g., sodium, potassium; salts of alkaline earth metals, e.g., calcium, magnesium, and barium; and salts of organic bases, e.g., amine bases and inorganic bases.

Compounds of the invention also include all isotopes of atoms occurring in the intermediate or final compounds. Isotopes include those atoms having the same atomic number but different mass numbers. For example, isotopes of hydrogen include tritium and deuterium. The term, “compound,” as used herein with respect to any inorganic nitrite or pharmaceutically acceptable salt thereof and is meant to include all stereoisomers, geometric iosorners, tautomers, and isotopes of the structures depicted. All compounds, and pharmaceutically acceptable salts thereof, are also meant to include solvated or hydrated forms.

The compounds of the present invention can be prepared in a variety of ways known to one of ordinary skill in the art of chemical synthesis. The compounds of the present invention can be synthesized using the methods described below, together with synthetic methods known in the art of synthetic chemistry or variations thereon as appreciated by one of ordinary skill in the art. Methods for preparing nitrite salts are well known in the art and a wide range of precursors and nitrite salts are readily available commercially. Nitrites of the alkali and alkaline earth metals can be synthesized by reacting a mixture of nitrogen monoxide (NO) and nitrogen dioxide (NO₂) with a corresponding metal hydroxide solution, as well as through the thermal decomposition of the corresponding nitrate. Other nitrites are available through the reduction of the corresponding nitrates.

The present compounds can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one of ordinary skill in the art by routine optimization procedures.

Regardless of their original source or the manner in which they are obtained, the compounds of the invention can be formulated in accordance with their use. For example, the compounds can be formulated within compositions for application to cells in tissue culture or for administration to a patient. When employed as nutritional supplement, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated.

Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes nutritional compositions which contain, as the active ingredient, one or more of the compounds described herein in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., nonnal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. As is known in the art, the type of diluent can vary depending upon the intended route of administration. The resulting compositions can include additional agents, such as preservatives. The compounds may also be applied to a surface of a device (e.g., a catheter) or contained within a pump, patch, or other drug delivery device. The therapeutic agents of the invention can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The pharmaceutical compositions can be formulated so as to provide quick, sustained or delayed release of the active ingredient after administration to the patient by employing procedures known in the art.

The compositions can be formulated in a unit dosage form, each dosage containing, for example, from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg” from about 0.5 mg to about 20 mg, from about 0.5 mg to about 10 mg, from about 0.5 mg to about 5 mg; from about 25 1 mg from to about 50 mg, from about 1 mg to about 30 mg” from about I mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient.

The term “unit dosage forms” refers to physically discrete units suitable as unitary dosages for human subjects and other mammals, each unit containing a predetermined quantity of active material calculated to produce the desired nutritional effect, in association with a suitable pharmaceutical excipient.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described herein and/or known in the art. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The compositions administered to a patient or a subject with nutritional deficiencies due to dietary restrictions can be in the form of one or more of the pharmaceutical compositions described above. These compositions can be sterilized by conventional sterilization techniques or may be sterile filtered. Aqueous solutions can be packaged for use as is, or lyophilized, the lyophilized preparation being combined with a sterile aqueous carrier prior to administration. The pH of the compound preparations typically will be between about 3 and 11, for example, between about 5 to 9, between 6 and 7, between 7 and 8. It will be understood that use of certain of the foregoing excipients, carriers, or stabilizers will result in the formation of pharmaceutical salts.

The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration.

Methods of Nutritional Supplementation

Nutritional deficiencies have been observed in patients with risk factors associated with cardiovascular diseases, peripheral artery disease patients and diabetic patients. These deficiencies are further pronounced in the latter subject groups following exercise. Other patients which may also benefit from nutritional supplementation with inorganic nitrite, or a pharmaceutically acceptable salt thereof, include those with chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, or peripheral neuropathy, stem cell diseases and patients with dietary restrictions. In each instance, the composition of this invention can be administered at a dose to supplement the nutritional deficiency but at which said dose does not increase the ration of methemoglobin:globin more than 1:10 in the blood.

The compositions of the invention are administered for a time and in an amount sufficient to result in the change in the gene expression pattern of genes involved in the inflammation process. Inflammation refers to all phases of the process of inflammation, including the initial signaling events, antigen presentation, dendritic cell activation, antibody production, T-cell activation, B-cell activation and cytokine expression. The inflammatory response may stem from any process that results in inflammation, for example, cancer, arthritis, infection or combinations thereof. The inflammatory response can by induced by an external stimulus, innate, or genetic.

Administration

The present methods for supplementing nutritional deficits are carried out by administering an inorganic nitrite for a time and in an amount sufficient to result in an increase in plasma nitrite or plasma nitric oxide levels, without increasing methemoglobin levels to more than about 10% of the total globin content. The amount and frequency of administration of the compositions can vary depending on, for example, what is being administered, the state of the patient's nutritional deficit, and the manner of administration. The dosage is likely to depend on such variables as the type and extent of nutritional deficit, the age, weight and general condition of the particular patient, the relative biological efficacy of the composition selected, formulation of the excipient, the route of administration, and the judgment of the attending clinician. Effective doses can be extrapolated from dose response curves derived from in vitro or animal model test system. An effective dose is a dose that produces a desirable increase in plasma levels of nitrite or nitric oxide without increasing the ration of methemoglobin:globin above I:10.

The amount of inorganic nitrite per dose can vary. For example, a subject can receive from about 0.05 μg/kg to about 1000 μg/kg., e.g., about 0.05, 1, 5, 10, 25, 50, 100, 200, 250, 300, 400, 450, 500, 550, 600, 650, 700, 750, 800, 850, 900, or 1000 μg/kg. Generally, we administer nitrite in an amount such that the circulating concentration does not exceed 0.6 μM, e.g., 0.05 μM, 0.1 μM, 0.15 μM, 0.2 μM, 0.25 μM, 0.3 μM, 0.35 μM, 0.4 μM, 0.45 μM, 0.5 μM, 0.55 μM, or 0.6 μM. Thus, exemplary dosages can include 8.25 μg/kg, 16.5 μg/kg or 165 μg/kg and exemplary circulating plasma concentrations can include 0.3 μM, 0.1 μM, or 0.05 μM.

The frequency of supplementation may also vary. The subject can be supplemented one or more times per day (e.g., once, twice, three, four or more times) or every so-many hours (e.g., about every 2, 4, 6, 8, 12, or 24 hours). The time course of supplementation may be of varying duration, e.g., for two, three, four, five, six, seven, eight, nine, ten or more days. For example, the supplementation can be twice a day for three days, twice a day for seven days, twice a day for ten days. Supplementation cycles can be repeated at intervals, for example weekly, bimonthly or monthly, which are separated by periods in which no supplement is given. The supplement can be a single supplement or can last as long as the life span of the subject (e.g., many years).

### Examples

The following example is provided to further illustrate certain aspects of the present invention and to aid those of skill in the art in practicing the invention, and is not meant to limit the scope of the invention.

The following ingredients are intimately mixed and loaded into a hard-shell capsule.

| | |
|---|---|
| Sodium Nitrite Powder | 3.500 gm |
| Benecel E4M ph cr (hypromellose) | 6.000 gm |
| Avicel ph-105 (microcrystalline cellulose) | 8.961 gm |
| Food color, yellow | 0.050 gm |
| fruit or herbal extract | 0.100 gm |

Modifications to the invention will be apparent to one of skill in the art given this disclosure. Such modifications and the resulting equivalents to the embodiments and example described above are intended to be included within the scope of the following claims.

1. A dietary supplement comprising one or more fruit or herbal extracts and at least 5% by weight of a nitrite salt.

2. The dietary supplement of claim 1, wherein the extract is an herbal extract selected form the group consisting of gingko biloba, garlic, ginger, pepper, parsley, orange, yohimbine, astragalus, catuaba, schisandra, and sulforaphane.

3. The dietary supplement of claim 2, wherein the herbal extract is a mixture of gingko biloba and garlic or wherein the herbal extract is gingko biloba.

4. (canceled)

5. The dietary supplement of claim 1 formulated for steady-state release over a period of about 2 hours to about 24 hours; formulated for daily administration; or formulated for oral administration.

6-7. (canceled)

8. The dietary supplement of claim 1, wherein the nitrite salt is sodium nitrite, potassium nitrite, or calcium nitrite.

9. The dietary supplement of claim 1 in the form of a tablet or capsule.

10. A method for increasing nitrite levels in a person having a nitrite nutritional deficiency, the method comprising administering to the person a supplement of claim 1 in a dosing regimen sufficient to alleviate the nutritional deficit.

11. The method of claim 10, wherein the dosing regimen administered to a subject results in a plasma level of nitrite of about 0.05 to 0.6 μM; wherein a unit dose of about 0.5 μg/kg to about 1000 μg/kg is administered; or wherein said nitrite nutritional deficiency is a result of dietary restrictions.

12-13. (canceled)

14. The method of claim 10, wherein said nitrite nutritional deficiency is a result of a disease state.

15. The method of claim 14, wherein said disease state is selected from the group consisting of diabetes, peripheral artery disease, chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, peripheral neuropathy, and stem cell diseases, or a combination of one or more of the disease states; wherein said disease state leads to decreased circulating levels of nitrite or nitric oxide; or wherein said disease state leads to decreased stored levels of nitrite.

16-17. (canceled)

18. The method of claim 10, further comprising the administration of a compound selected from a sulfide, clopidogrel, or dipyridamole.

19. An oral pharmaceutical composition for treating nitrite deficiency in a subject, which composition comprises: a sufficient amount of a pharmaceutically acceptable nitrite salt so that upon administration an increased serum nitrite concentration of from about 200 nM to about 1000 nM of nitrite is achieved.

20. The oral pharmaceutical composition of claim 19 further comprising an enteric coating.

21. The oral pharmaceutical composition of claim 20 wherein the enteric coating serves to release the nitrite salt to achieve the increased serum nitrite concentration over a period of at least 8 hours, at least 6 hours, at least 4 hours, or at least 2 hours.

22-24. (canceled)

25. The oral pharmaceutical composition of claim 19 for increasing serum nitrite concentrations to at least 300 nM or to at least 400 nm.

26. (canceled)

27. The oral pharmaceutical composition of claim 19 wherein the pharmaceutically acceptable nitrite salt is selected from the group consisting of sodium nitrite, potassium nitrite, and calcium nitrite, or mixtures thereof.

28. The oral pharmaceutical composition of claim 19, further comprising a compound selected from a sulfide, clopidogrel, or dipyridamole.

29. A method for treating nitrite deficiency in a subject, which method comprises administering to the subject the oral pharmaceutical composition of claim 19.

30. The method of claim 29 wherein the oral pharmaceutical composition is administered to the subject in a pharmaceutically acceptable nitrite salt to subject weight/weight ratio of from about 0.1 to about 2 mg/kg.

31. The method of claim 30 wherein treatment is continued until the tissue concentration of nitrite in the patient reaches between 300 nM and 1000 nM.

32. The composition of claim 19, wherein said composition is administered in a form to provide a steady-state release over a period of about 2 hours to about 24 hours.

33. A method for supplementing nutritional deficiencies in a patient or person in need thereof, comprising administering to a patient or a person a composition of inorganic nitrite or a pharmaceutically acceptable salt thereof, wherein the nitrite is administered for a time and in an amount sufficient to correct the nutritional deficits found in subjects.

34. The method of claim 33, wherein the dose administered to a patient results in a plasma level of nitrite of about 0.1 to 1.0 μM, preferably, 0.3 μM, and which does not lead to an increase in the ratio of methemoglobin:globin of more than 1:10 in said patient or wherein the dose is about 100 μg/kg to about 2000 μg/kg.

35. (canceled)

36. The method of claim 33, wherein said composition is administered to said patient daily.

37. The method of claim 33, wherein said composition is administered to said patient orally, sublingually, topically, intramuscularly, intraperitoneally, intravenously, or subcutaneously.

38. (canceled)

39. The method of claim 33, wherein said composition is administered in a form to provide a steady-state release over a period of about 2 hours to about 24 hours.

40. The method of claim 33, wherein said composition further comprises a pharmaceutically acceptable carrier.

41. The method of claim 33, wherein the pharmaceutically acceptable salt of inorganic nitrite is sodium nitrite, potassium nitrite, or calcium nitrite.

42. The method of claim 33, wherein said nutritional deficiencies are a result of dietary restrictions or wherein said nutritional deficiencies are a result of a disease state.

43. (canceled)

44. The method of claim 42 wherein said disease state is selected from one or more of the group consisting of diabetes, peripheral artery disease, chronic infections, acute infections, congestive heart failure, atherosclerotic cardiovascular disease, intermittent claudication, critical limb ischemic disease, defective wound healing, stroke, myocardial infarction, inflammatory bowel disease, a bone fracture, a bone infection, or peripheral neuropathy and stem cell diseases.

45. The method of claim 42, wherein said disease state leads to decreased circulating levels of nitrite in said patient; wherein said disease state leads to decreased stored levels of nitrite in said patient; or wherein said disease states leads to decreased circulating levels of nitric oxide in said subject.

46-48. (canceled)

49. The method of claim 29, wherein said method comprises the administration of a compound selected from a sulfide, clopidogrel, or dipyridamole.
